(19) United States
(12) Patent Application Publication
(10) Pub. No.: US 2012/0238877 A1
(43) Pub. Date: Sep. 20, 2012

(54) ULTRASOUND DIAGNOSTIC APPARATUS AND ULTRASOUND IMAGE PRODUCING METHOD

(75) Inventor: Rika TASHIRO, Ashigara-kami-gun (JP)

(73) Assignee: FUJIFILM CORPORATION, Tokyo (JP)

(21) Appl. No.: 13/359,162

(22) Filed: Jan. 26, 2012

(30) Foreign Application Priority Data
Mar. 17, 2011 (JP) 2011-059175

(51) Int. Cl. A61B 8/14

(52) U.S. Cl. 600/447

(57) ABSTRACT

An ultrasound diagnostic apparatus includes: a convex type ultrasound probe including a transducer array with a curvature; an image producer for producing a B mode image based on reception data; an abdominal wall detector for detecting an abdominal wall of the subject on the B mode image; a controller for obtaining reception data for a sound speed map by transmitting and receiving ultrasonic beams for a sound speed map through the transducer array by convex scan when a shape of the abdominal wall substantially corresponds to the curvature of the transducer array and by transmitting and receiving ultrasonic beams for a sound speed map through the transducer array by linear scan when the shape of the abdominal wall is substantially linear; and a sound speed map producer for producing a sound speed map based on the obtained reception data for a sound speed map.

### BACKGROUND OF THE INVENTION

The present invention relates to an ultrasound diagnostic apparatus and an ultrasound image producing method and particularly to an ultrasound diagnostic apparatus that produces both a B mode image and a sound speed map by transmitting and receiving ultrasonic waves through a transducer array of an ultrasound probe.

Conventionally, ultrasound diagnostic apparatus using ultrasound images are employed in medicine. In general, this type of ultrasound diagnostic apparatus comprises an ultrasound probe having a built-in transducer array and an apparatus body connected to the ultrasound probe. The ultrasound probe transmits an ultrasonic beam toward the inside of a subject's body, receives ultrasonic echoes from the subject, and the apparatus body electrically processes the reception signals to produce an ultrasound image.

In recent years, sound speeds in a region under examination are measured to achieve a more accurate diagnosis of the region inside the subject's body.

JP 2010-99452 A, for example, proposes an ultrasound diagnostic apparatus whereby a plurality of lattice points are set around a site under examination and an ultrasonic beam is transmitted to and received from the lattice points to obtain reception data, based on which local sound speeds are calculated.

JP 2010-99452 A describes a device having an ultrasound probe that transmits and receives an ultrasonic beam to and from the inside of a subject's body to obtain local sound speeds at a site under examination, thereby enabling display of a B mode image with, for example, the local sound speeds superimposed over it. Further, producing a sound speed map representing a distribution of local sound speeds at respective points in a given region and displaying it together with the B mode image effectively support diagnosis of a site under examination.

Known ultrasound probes include ones of convex type, sector type, and linear type according to scan methods. In screening, for example, a convex type ultrasound probe is commonly used in that it enables a wide-angle observation region to be readily obtained. The convex type ultrasonic beam has a plurality of ultrasound transducers constituting a transducer array arranged in the form of a sector, so that a plurality of ultrasonic beams are radially transmitted from the transducer array by convex scan.

However, because the sound speed at a point near the abdominal wall covering organs, for examples, is different from that in other points because of the existence of fat among other causes, radial transmission of ultrasonic beams from the transducer array of the convex type ultrasound probe in an attempt to obtain local sound speeds in a site under examination increases the effects of refraction caused by the abdominal wall as the ultrasonic beams pass through the abdominal wall, possibly making it impossible to produce an accurate sound speed map.

In addition, there arises another problem that radial transmission of ultrasonic beams causes the measuring accuracy to decrease as the measuring depth increases.

### SUMMARY OF THE INVENTION

An object of the present invention is to eliminate such problems associated with the prior art and provide an ultrasound diagnostic apparatus and an ultrasound image producing method capable of producing a B mode image and producing an accurate sound speed with a convex type ultrasonic probe with reducing effects of refraction of ultrasonic beams caused by an abdominal wall.

An ultrasound diagnostic apparatus according to the present invention comprises:

a convex type ultrasound probe including a transducer array with a curvature;

a transmission circuit for transmitting ultrasonic beams from the transducer array toward a subject;

a reception circuit for processing reception signals outputted from the transducer array having received ultrasonic echoes from the subject to obtain reception data;

an image producer for producing a B mode image based on the reception data obtained by the reception circuit;

an abdominal wall detector for detecting an abdominal wall of the subject on the B mode image produced by the image producer;

a controller for controlling the transmission circuit and the reception circuit to transmit and receive the ultrasonic beams for a sound speed map through the transducer array by convex scan to obtain reception data for a sound speed map when a shape of the abdominal wall detected by the abdominal wall detector substantially corresponds to the curvature of the transducer array, and controlling the transmission circuit and the reception circuit to transmit and receive the ultrasonic beams for a sound speed map through the transducer array by linear scan to obtain reception data for a sound speed map when the shape of the abdominal wall detected by the abdominal wall detector is substantially linear; and

a sound speed map producer for producing a sound speed map based on the obtained reception data for a sound speed map.

A method of producing an ultrasound image according to the present invention comprises the steps of:

producing a B mode image based on reception data obtained by transmitting and receiving ultrasonic beams through a transducer array with a curvature of a convex type ultrasound probe to and from a subject and processing reception signals outputted from the transducer array having received ultrasonic echoes from the subject;

detecting an abdominal wall of the subject on the B mode image;

obtaining reception data for a sound speed map by transmitting and receiving ultrasonic beams for a sound speed map through the transducer array by convex scan when a shape of a detected abdominal wall substantially corresponds to the curvature of the transducer array and by transmitting and receiving ultrasonic beams for a sound speed map from the transducer array by linear scan when the shape of the detected abdominal wall is substantially linear; and

producing a sound speed map based on the obtained reception data for a sound speed map.

### BRIEF DESCRIPTION OF THE DRAWINGS

FIG. 1 is a block diagram illustrating a configuration of an ultrasound diagnostic apparatus according to an embodiment of the invention.

FIG. 2 schematically illustrates a B mode image.

FIGS. 3A and 3B schematically illustrate a principle of sound speed calculation according to the embodiment.

FIG. 4 is a flow chart illustrating the operation of the embodiment.

FIG. 5 illustrates ultrasonic beams for a sound speed map in a convex-scanning.

FIG. 6 illustrates ultrasonic beams for a sound speed map in a linear-scanning.

### DETAILED DESCRIPTION OF THE INVENTION

An embodiment of the present invention will now be described below based on the appended drawings.

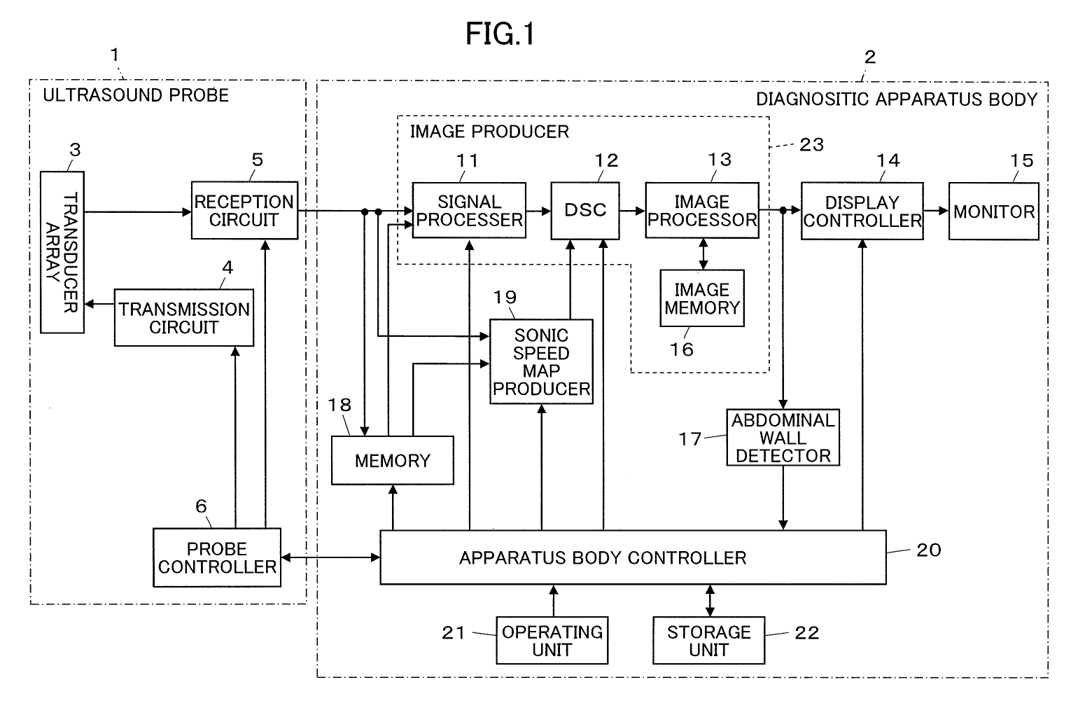

FIG. 1 illustrates a configuration of an ultrasound diagnostic apparatus according to the embodiment of the invention. The ultrasound diagnostic apparatus comprises an ultrasound probe 1 and a diagnostic apparatus body 2 connected to the ultrasound probe 1.

The ultrasound probe 1 is a so-called convex type probe, where a transducer array 3 is connected to a transmission circuit 4 and a reception circuit 5, which in turn are connected to a probe controller 6.

The diagnostic apparatus body 2 comprises a signal processor 11 connected to the reception circuit 5 of the ultrasound probe 1. The signal processor 11 is connected in sequence to a DSC (Digital Scan Converter) 12, an image processor 13, a display controller 14, and a monitor 15. The image processor 13 is connected to an image memory 16 and an abdominal wall detector 17. The diagnostic apparatus body 2 further comprises a memory 18 and a sound speed map producer 19 connected to the reception circuit 5 of the ultrasound probe 1. The signal processor 11, the DSC 12, the display controller 14, the abdominal wall detector 17, the memory 18, and the sound speed map producer 19 are connected to an apparatus body controller 20. The apparatus body controller 20 is connected to an operating unit 21 and a storage unit 22.

The probe controller 6 of the ultrasound probe 1 and the apparatus body controller 20 of the diagnostic apparatus body 2 are connected to each other.

The transducer array 3 of the ultrasound probe 1 comprises a plurality of ultrasound transducers arranged in the form of a sector and has an outwardly curved shape having a given curvature. The ultrasound transducers of the transducer array 3 each transmit ultrasonic waves according to actuation signals supplied from the transmission circuit 4 and receive ultrasonic echoes from the subject to output reception signals. Each of the ultrasound transducers comprises an oscillator composed of a piezoelectric body and electrodes each provided on both ends of the piezoelectric body. The piezoelectric body is composed of, for example, a piezoelectric ceramic typified by a PZT (titanate zirconate lead), a polymeric piezoelectric device typified by PVDF (polyvinylidene flouride), or a piezoelectric monochristal typified by PMN-PT (lead magnesium niobate lead titanate solid solution).

When the electrodes of each of the oscillators are supplied with a pulsed voltage or a continuous-wave voltage, the piezoelectric body expands and contracts to cause the oscillator to produce pulsed or continuous ultrasonic waves. These ultrasonic waves are combined to form an ultrasonic beam. Upon reception of propagating ultrasonic waves, each oscillator expands and contracts to produce an electric signal, which is then outputted as reception signal for the ultrasonic waves.

The transmission circuit 4 includes, for example, a plurality of pulsers and adjusts the delay amounts for actuation signals based on a transmission delay pattern selected according to a control signal transmitted from the probe controller 6 so that the ultrasonic waves transmitted from a plurality of ultrasound transducers of the transducer array 3 form an ultrasonic beam, and supplies the ultrasound transducers with delay-adjusted actuation signals.

The reception circuit 5 amplifies and A/D-converts the reception signals transmitted from the ultrasound transducers of the transducer array 3, and then performs reception focusing processing by providing the reception signals with respective delays according to the sound speed or sound speed distribution that is set based on a reception delay pattern selected according to the control signal transmitted from the probe controller 6 and adding them up. This reception focusing processing yields reception data (sound ray signals) having the ultrasonic echoes well focused.

The probe controller 6 controls various components of the ultrasound probe 1 according to control signals transmitted from the apparatus body controller 20 of the diagnostic apparatus body 2.

The signal processor 11 of the diagnostic apparatus body 2 corrects attenuation in the reception data produced by the reception circuit 6 of the ultrasound probe 1, the attenuation depending on the distance that varies with the depth at which the ultrasonic waves are reflected, and then performs envelope detection processing to produce a B mode image signal, which is tomographic image information on a tissue inside the subject's body.

The DSC 12 converts the B mode image signal produced by the signal processor 11 into an image signal compatible with an ordinary television signal scan mode (raster conversion).

The image processor 13 performs various processing required including gradation processing on the B mode image signal entered from the DSC 12 before outputting the B mode image signal to the display controller 14 or storing the B mode image signal in the image memory 16.

The signal processor 11, the DSC 12, the image processor 13, and the image memory 16 constitute an image producer 23.

The display controller 14 causes the monitor 15 to display an ultrasound diagnostic image based on the B mode image signal having undergone image processing by the image processor 13.

The monitor 15 includes a display device such as an LCD, for example, and displays an ultrasound diagnostic image under the control of the display controller 14.

According to a B mode image signal that was image-processed by the image processor 13, the abdominal wall detector 17 detects a subject's abdominal wall P located above a region of interest ROI set in the B mode image.

The memory 18 sequentially stores the reception data outputted from the reception circuit 5 of the ultrasound probe 1. The memory 18 stores information on a frame rate entered from the apparatus body controller 20 in association with the above reception data. Such information includes, for example, the depth of a position at which the ultrasonic waves are reflected, the density of scan lines, and a parameter representing the range of the visual field.

Under the control by the apparatus body controller 20, the sound speed map producer 19 calculates the local sound speeds in a tissue inside the subject's body under examination based on the reception data stored in the memory 18 to produce the sound speed map.

The apparatus body controller 20 controls the components in the ultrasound diagnostic apparatus according to the instructions entered by the operator using the operating unit 21.

The operating unit 21, provided for the operator to perform input operations, constitutes a region-of-interest setting unit and may be composed of, for example, a keyboard, a mouse, a track ball, and/or a touch panel.

The storage unit 22 stores, for example, an operation program and may be constituted by, for example, a recording medium such as an MO, an MT, a RAM, a CD-ROM, a DVD-ROM, an SD card, a CF card, or a USB memory, or a server.

Although the signal processor 11, the DSC 12, the image processor 13, the display controller 14, and the sound speed map producer 19 are each constituted by a CPU and an operation program for causing the CPU to perform various kinds of processing, they may be each constituted by digital circuits.

The operator may select one of the following three display modes using the operating unit 21. They are: a mode for displaying the B mode image alone, a mode for displaying the B mode image, with the sound speed map superimposed over it (e.g., display with color distinction or by varying luminance according to the local sound speed, or display where points having an equal local sound speed are connected by a line), and a mode for displaying the B mode image and the sound speed map image in juxtaposition. The B mode image may be displayed in a desired mode selected from these modes.

When the B mode image is displayed, firstly a plurality of ultrasound transducers of the transducer array 3 transmit ultrasonic waves according to the actuation signals supplied from the transmission circuit 4 of the ultrasound probe 1, and the ultrasound transducers having received ultrasonic echoes from the subject output the reception signals to the reception circuit 5, which produces the reception data. The signal processor 11 of the diagnostic apparatus body 2 having received the reception data produces the B mode image signal, and the DSC 12 performs raster conversion of the B mode image signal, while the image processor 13 performs various image processing on the B mode image signal, whereupon, based on this B-mode image signal, the display controller 14 causes the monitor 15 to display the ultrasound diagnostic image.

The local sound speed may be calculated by, for example, a method described in JP 2010-99452 A filed by the Applicant of the present application.

Suppose, as illustrated in FIG. 3A, that, on transmission of ultrasonic waves to the inside of a subject, reception waves Wx reach the transducer array 1 from the lattice point X, a reflection point in the subject, and that a plurality of lattice points A1, A2, . . . are arranged at equal intervals in positions shallower than the lattice point X, i.e., in positions closer to the transducer array 1, as illustrated in FIG. 3B. Then, the local sound speed at the lattice point X is obtained according to the Huygens principle whereby a synthesized wave Wsum produced by combining individual reception waves W1, W2, . . . transmitted from the lattice points A1, A2, . . . having received a reception signal from the lattice point X coincides with the reception waves Wx from the lattice point X.

First, optimum sound speeds for all the lattice points X, A1, A2, . . . are obtained. The optimum sound speed herein means a sound speed allowing a highest image contrast and sharpness to be obtained as a set sound speed is varied after performing focus calculation for the lattice points based on the set sound speed and imaging to produce an ultrasound image. The optimum sound speed may be judged based on, for example, the image contrast, spatial frequency in the scan direction, and dispersion as described in JP 08-317926 A.

Next, the optimum sound speed for the lattice point X is used to calculate the waveform of an imaginary reception waves Wx emitted from the lattice point X.

Further, a hypothetical local sound speed V at the lattice point X is changed to various values to calculate the imaginary synthesized wave Wsum of the reception waves W1, W2, . . . from the lattice points A1, A2, . . . Suppose that, at this time, the sound speed is consistent in a region Rxa between the lattice point X and the lattice points A1, A2, . . . and is equivalent to the local sound speed V at the lattice point X. The times in which the ultrasonic wave propagating from the lattice point X reaches the lattice points A1, A2, . . . are XA1/V, XA2/V, . . . , respectively, where XA1, XA2, . . . are the distances between the lattice point X and the lattice points A1, A2, . . . Combining the reflected waves emitted from the lattice points A1, A2, . . . with respective delays corresponding to the times XA1/V, XA2/V, . . . yields the imaginary synthesized wave Wsum.

Next, the respective differences between a plurality of the imaginary synthesized waves Wsum calculated by changing the hypothetical local sound speed V at the lattice point X to various values and the imaginary reception waves Wx from the lattice point X are calculated to determine the hypothetical local sound speed V at which the difference becomes a minimum as the local sound speed. The difference between the imaginary synthesized waves Wsum and the imaginary reception waves Wx from the lattice point X may be calculated by any of appropriate methods including a method using the cross-correlation, a method using phase matching addition by multiplying the reception waves Wx by a delay obtained from the synthesized wave Wsum, and a method using phase matching addition by multiplying the synthesized wave Wsum by a delay obtained from the reception waves Wx.

Thus, the local sound speeds inside a subject can be accurately calculated based on the reception data produced by the reception circuit 5 of the ultrasound probe 1. The sound speed map representing a distribution of the local sound speeds in a set region of interest may be likewise produced.

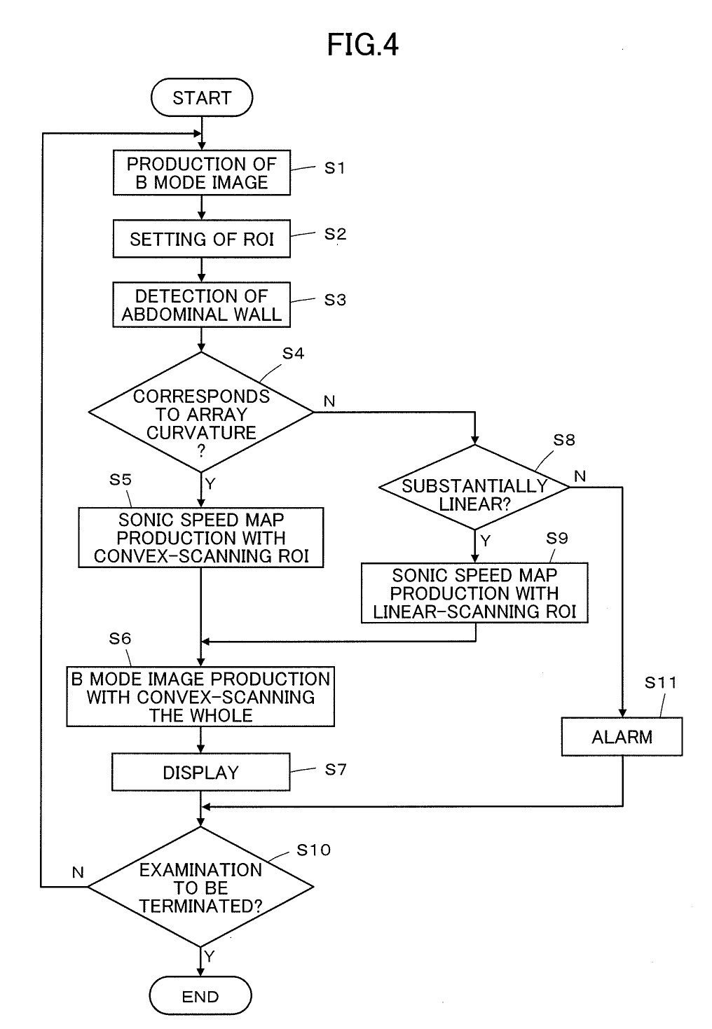

Next, the operation of the embodiment will be described referring to the flowchart of FIG. 4.

First, in step S1, according to the actuation signal from the transmission circuit 4 of the ultrasound probe 1, a plurality of ultrasound transducers of the transducer array 3 transmit an ultrasonic beam for the B mode image, and the ultrasound transducers that have received ultrasound echoes from a subject output reception signals to the reception circuit 5 to produce reception data for the B mode image, whereupon the display controller 14 causes the monitor 15 to display the B mode image based on the B mode image signal produced by the image producer 23 of the diagnostic apparatus body 2.

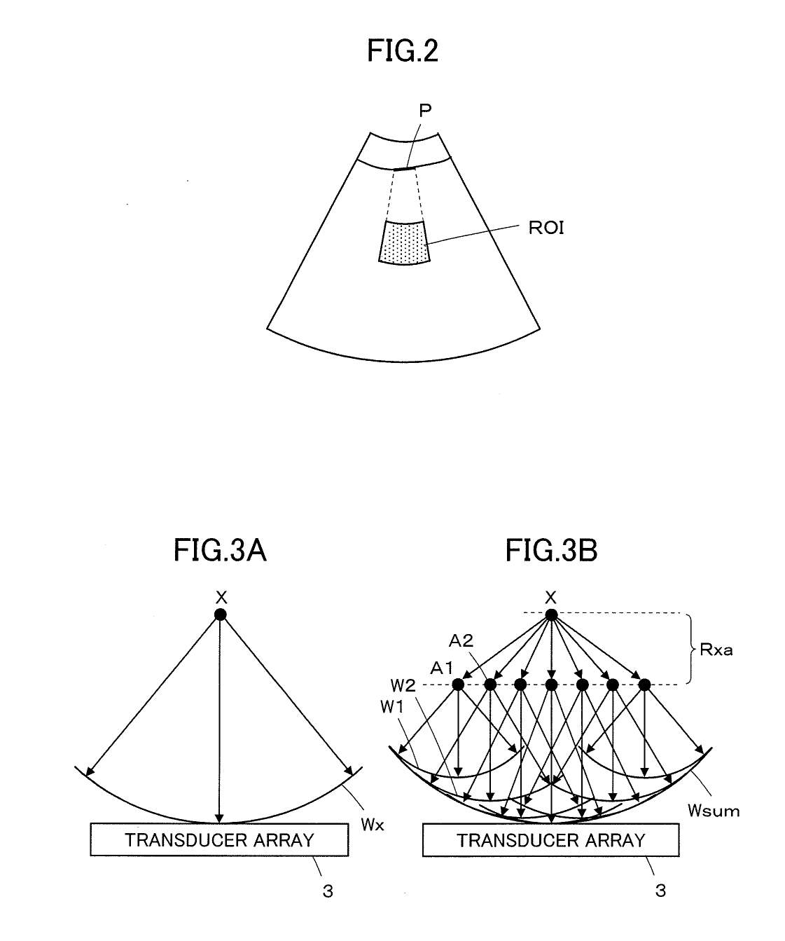

After the operator operates the operating unit 21 to set the region of interest ROI in the B mode image displayed on the monitor 15 in step S2, the abdominal wall detector 17 detects the subject's abdominal wall P located above the region of interest ROI as illustrated in FIG. 2 in step S3. Thereafter, the apparatus body controller 20 compares the shape of the subject's abdominal wall P detected by the abdominal wall detector 17 with the curvature of the transducer array 3 of the ultrasound probe 1 in step S4.

When the shape of the abdominal wall P is judged to substantially correspond to the curvature of the transducer array 3 to within a given tolerance, the procedure proceeds to step 5, where the ultrasonic beams for the sound speed map emitted from the transducer array are transmitted and received as they convex-scan the region of interest ROI to produce a sound speed map of the inside of the region of interest RIO.

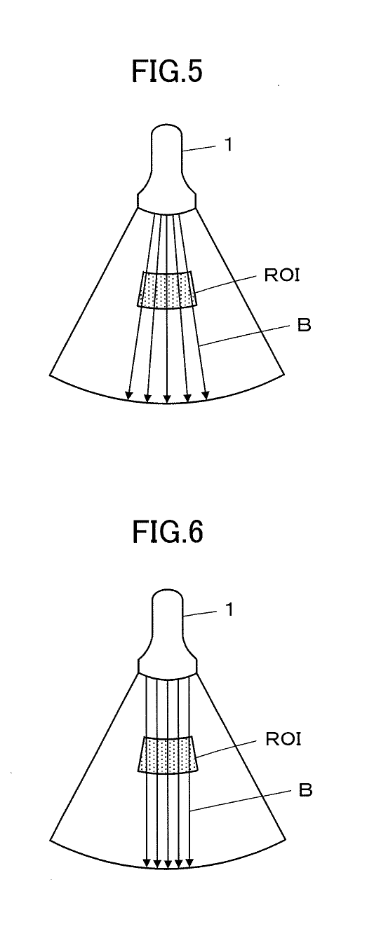

Specifically, a plurality of lattice points are set in the region of interest ROI by the apparatus body controller 20, and a transmission focus is formed at these lattice points, so that ultrasonic beams for the sound speed map are sequentially transmitted and received as they convex-scan the region of interest. Because the shape of the abdominal wall P substantially corresponds to the curvature of the transducer array 3, ultrasonic beams B for the sound speed map radially transmitted from the transducer array 3 enter the abdominal wall P substantially at right angles as illustrated in FIG. 5 and are virtually spared the effects of refraction caused by the abdominal wall P as they pass through the abdominal wall P, forming the transmission focus at the lattice points in the region of interest ROI. Then, the ultrasonic echoes from the subject are received by the ultrasound transducers of the transducer array 3.

Each time the ultrasonic beam for the sound speed map is received, the reception data for the sound speed map produced by the reception circuit 5 are sequentially stored in the memory 18. When the reception data for the sound speed map have been obtained for all the lattice points in the region of interest ROI, the apparatus body controller 20 outputs an instruction for producing the sound speed map to the sound speed map producer 19, which then uses the reception data for the sound speed map stored in the memory 18 to calculate local sound speeds at the lattice points and produce the sound speed map of the inside of the region of interest ROI. The data on the sound speed map obtained by the sound speed map producer 19 undergo raster conversion through the DSC 12 and various image processing by the image processor 13.

When, on the other hand, the comparison made in step S4 shows that the shape of the abdominal wall P does not substantially correspond to the curvature of the transducer array 3 to within a given tolerance, the procedure proceeds to step S8 to judge whether the shape of the abdominal wall P is substantially linear or not. When the shape of the abdominal wall P is judged to be substantially linear, the procedure proceeds to step 9, where the ultrasonic beams for the sound speed map emitted from the transducer array are transmitted and received as they linear-scan the region of interest ROI to produce a sound speed map of the inside of the region of interest RIO.

Specifically, a plurality of lattice points are set in the region of interest ROI by the apparatus body controller 20, and the transmission focus is formed at these lattice points, so that ultrasonic beams for the sound speed map are sequentially transmitted and received as they linear-scan the region of interest. Because the shape of the abdominal wall P is substantially linear, the ultrasonic beams B for the sound speed map transmitted in parallel from the transducer array 3 enter the abdominal wall P substantially at right angles as illustrated in FIG. 6 and are virtually spared the effects of refraction caused by the abdominal wall P as they pass through the abdominal wall P, forming the transmission focus at the lattice points in the region of interest ROI. Then, the ultrasonic echoes from the subject are received by the ultrasound transducers of the transducer array 3.

Each time the ultrasonic beam for the sound speed map is received, the reception data for the sound speed are sequentially stored in the memory 18, so that the sound speed map producer 19 produces the sound speed map of the inside of the region of interest ROI in the same manner as by the convex scan in step S5 described above. The data on the sound speed map obtained by the sound speed map producer 19 undergo raster conversion through the DSC 12 and various image processing by the image processor 13.

Upon production of the sound speed map of the region of interest ROI in step S5 or step S9 as described above, the ultrasonic beams for the B mode image emitted from the transducer array 3 are transmitted and received as they convex-scan the whole imaging region to produce the B mode image in step S6.

Specifically, the ultrasound traducers having received the ultrasonic echoes of the ultrasonic beams for the B mode image transmitted from the transducer array 3 output the reception signals to the reception circuit 5 to produce the reception data for the B mode image, and the reception data for the B mode image are stored in the memory 18 of the diagnostic apparatus body 2 and inputted into the signal processor 11 to produce the B mode image signal, whereupon the B mode image signal undergoes raster conversion through the DSC 12 and various image processing through the image processor 13.

In step S7, the data on the sound speed map of the inside of the region of interest ROI and the B mode image having undergone various image processing through the image processor 13 are transmitted to the display controller 14 and displayed on the monitor 15 with the sound speed map superimposed on the B mode image or displayed on the monitor 15, with the B mode image and the sound speed map arranged in juxtaposition with each other, depending on the display mode entered by the operator using the operating unit 21.

In step S10, whether the examination is to be terminated or not is determined. When the examination is to be continued, the procedure returns to step S1, while when the examination is to be terminated, the sequential processing is terminated.

When the shape of the abdominal wall P is judged not to be substantially linear in step S8, the shape of the abdominal wall P does not substantially corresponds to the curvature of the transducer array 3 and is not substantially linear. Therefore, the angle of incidence at which the ultrasonic beams B for the sound speed map enter the abdominal wall P increases and hence is greatly affected by the refraction caused by the abdominal wall P whether the scan by the ultrasonic beams B for the sound speed map emitted from the transducer array 3 of the convex type ultrasound probe 1 as the beam is transmitted and received is the convex scan or the linear scan. Accordingly, the transmission circuit 4 and the reception circuit 5 are controlled not to transmit or receive of the ultrasonic beams B for the sound speed map because an accurate sound speed map of the inside of the region of interest ROI cannot be produced, so that an alarm is given on the monitor 15, for example, before proceeding to step S10 to determine whether the examination is to be terminated or not.

Judgment in step S4 as to whether the shape of the abdominal wall P substantially corresponds to the curvature of the transducer array 3 within a given tolerance or not may be made, for example, as follows.

First, a plurality of measuring points Qi (i=1 to n) are set on the abdominal wall P detected by the abdominal wall detector 17. The coordinates (Xi, Yi) of these measuring points Qi on an image are used to obtain an approximate curve having the same curvature as the transducer array 3 by the method of least squares and calculate a coefficient of variation CV of residuals dYi of the measuring points Qi.

Let the mean value of the residuals dYi of the measuring points Qi be dYm. Then, the coefficient of variation CV of the residuals dYi is expressed as

CV=[(1/n)Σ(dYi²−dYm²)]^{1/2}/dYm (1)

where Σ denotes the total sum for i=1 to n.

Then, with a threshold CV1 set to, for example, 0.1 to determine a tolerance, the shape of the abdominal wall P may be judged to substantially correspond to the curvature of the transducer array 3 when the coefficient of variation CV of the residuals dYi calculated using the above expression (1) is not greater than the threshold CV1, whereas the shape of the abdominal wall P may be judged not to substantially correspond to the curvature of the transducer array 3 when the coefficient of variation CV is greater than the set value CV1.

Judgment in step S8 as to whether the shape of the abdominal wall P is substantially linear or not may be made, for example, as follows.

A plurality of measuring points Qi (i=1 to n) are set on the abdominal wall P detected by the abdominal wall detector 17. The coordinates (Xi, Yi) of these measuring points Qi on an image are used to obtain an approximate line by the method of least squares and calculate the coefficient of variation CV of the residuals dYi of the measuring points Qi using the above expression (1).

When the coefficient of variation CV of the calculated residuals dYi is not greater than the threshold CV1=0.1, the shape of the abdominal wall P is judged to be substantially linear, and when the coefficient of variation CV is greater than the set value CV1, the shape of the abdominal wall P is judged not to be substantially linear.

The above threshold CV1 is not limited to “0.1”. It is preferable to obtain a threshold CV1 such that the disturbance in wavefront does not adversely affect the measuring of the sound speed by actually transmitting and receiving the ultrasonic beam for the sound speed map by the convex scan.

Judgment in step S8 as to whether the shape of the abdominal wall P is substantially linear or not may also be made using a correlation coefficient.

That is, a plurality of measuring points Qi (i=1 to n) are set on the abdominal wall P detected by the abdominal wall detector 17, and a correlation coefficient r of the coordinates (Xi, Yi) of these measuring points Qi on an image is calculated

Let Xm be a mean value of Xm, and Ym a mean value of Yi. Then, the correlation coefficient r is expressed as

r=Σ[(Xi−Xm) (Yi−Ym) ]/[Σ(Xi−Xm) ²Σ(Yi−Ym)²]^{1/2} (2)

where Σ denotes the total sum for i=1 to n.

Then, with a threshold r1 set to, for example, 0.7 to determine a tolerance, the shape of the abdominal wall P is judged to be substantially linear when the absolute value of the correlation coefficient r calculated using the above expression (2) is not smaller than the threshold r1, whereas the shape of the abdominal wall P is judged not to be substantially linear when the absolute value of the correlation coefficient r is smaller than the threshold r1.

Also in this case, the threshold r1 is not limited to “0.7” and is preferably set to a value appropriate for the actual measuring.

Thus, whether the shape of the abdominal wall P detected by the abdominal wall detector 17 substantially corresponds to the curvature of the transducer array 3 or is substantially linear is judged, and depending on the judgment made, the ultrasonic beams B for the sound speed map are transmitted and received by the convex scan. Therefore, even by using the convex type ultrasound probe 1, the effects of refraction caused by the abdominal wall P can be reduced, and production of the B mode image and an accurate sound speed map are made possible.

Although, in the above embodiment, when the shape of the abdominal wall P neither substantially corresponds to the curvature of the transducer array 3 nor is substantially linear, the transmission circuit 4 and the reception circuit 5 are controlled not to allow transmission and reception of the ultrasonic beam B for the sound speed map, the sound speed map may be alternatively produced as follows when the shape of the abdominal wall P substantially has an intermediate curvature between the curvature of the transducer array 3 and a substantial linearity.

The apparatus body controller 20 calculates a delay corresponding to the curvature of the abdominal wall P detected by the abdominal wall detector 17, the calculated delay is transmitted via the probe controller 6 to the transmission circuit 4 and the reception circuit 5, whereupon the transmission circuit 4 and the reception circuit 5 are so controlled as to ensure that the ultrasonic beams B for the sound speed map are transmitted by the convex scan with the calculated delay to obtain the reception data for the sound speed map.

Thus, the ultrasonic beams B for the sound speed map adapted to suit the curvature of the abdominal wall P can be transmitted and received by the convex scan, and hence the effects of refraction caused by the abdominal wall P can be reduced and an accurate sound speed map can be produced even when the abdominal wall P has a different curvature from that of the transducer array 3.

While, in the above embodiments, the reception data outputted from the reception circuit 5 are first stored in the memory 18, so that the sound speed map producer 19 uses the reception data stored in the memory 18 to calculate the local sound speeds at the lattice points in the region of interest ROI and produce the sound speed map of the inside of the region of interest ROI, the sound speed map producer 19 may directly receive the reception data outputted from the reception circuit 5 to produce the sound speed map.

Because the memory 18 stores not only the reception data for the sound speed map but the reception data for producing the B mode image, the reception data for producing the B mode image may be read from the memory 18 as necessary by the control given by the apparatus body controller 20 for the image producer 23 to produce the B mode image.

The connection between the ultrasound probe 1 and the diagnostic apparatus body 2 may be achieved by wired communication or wireless communication.

What is claimed is:

1. An ultrasound diagnostic apparatus comprising:
a convex type ultrasound probe including a transducer array with a curvature;
a transmission circuit for transmitting ultrasonic beams from the transducer array toward a subject;
a reception circuit for processing reception signals outputted from the transducer array having received ultrasonic echoes from the subject to obtain reception data;
an image producer for producing a B mode image based on the reception data obtained by the reception circuit;
an abdominal wall detector for detecting an abdominal wall of the subject on the B mode image produced by the image producer;
a controller for controlling the transmission circuit and the reception circuit to transmit and receive the ultrasonic beams for a sound speed map through the transducer array by convex scan to obtain reception data for a sound speed map when a shape of the abdominal wall detected by the abdominal wall detector substantially corresponds to the curvature of the transducer array, and controlling the transmission circuit and the reception circuit to transmit and receive the ultrasonic beams for a sound speed map through the transducer array by linear scan to obtain reception data for a sound speed map when the shape of the abdominal wall detected by the abdominal wall detector is substantially linear; and
a sound speed map producer for producing a sound speed map based on the obtained reception data for a sound speed map.

2. The ultrasound diagnostic apparatus according to claim 1,
further comprising a region-of-interest setting unit for setting a region of interest in the B mode image produced by the image producer,
the abdominal wall detector detecting an abdominal wall of the subject located above the region of interest set by the region-of-interest setting unit.

3. The ultrasound diagnostic apparatus according to claim 1,
wherein the controller controls the transmission circuit and the reception circuit not to perform transmission and reception of ultrasonic beams for a sound speed map when the shape of the abdominal wall detected by the abdominal wall detector does not corresponds to the curvature of the transducer array and is not substantially linear.

4. the ultrasound diagnostic apparatus according to claim 1,
wherein, when the shape of the abdominal wall detected by the abdominal wall detector has an intermediate curvature between the curvature of the transducer array and a substantial linearity, the controller calculates a delay corresponding to the curvature of the abdominal wall and controls the transmission circuit and the reception circuit to transmit and receive the ultrasonic beams for a sound speed map through the transducer array by convex scan with the calculated delay to obtain reception data for a sound speed map.

5. A method of producing an ultrasound image comprising the steps of:
producing a B mode image based on reception data obtained by transmitting and receiving ultrasonic beams through a transducer array with a curvature of a convex type ultrasound probe to and from a subject and processing reception signals outputted from the transducer array having received ultrasonic echoes from the subject;
detecting an abdominal wall of the subject on the B mode image;
obtaining reception data for a sound speed map by transmitting and receiving ultrasonic beams for a sound speed map through the transducer array by convex scan when a shape of a detected abdominal wall substantially corresponds to the curvature of the transducer array and by transmitting and receiving ultrasonic beams for a sound speed map from the transducer array by linear scan when the shape of the detected abdominal wall is substantially linear; and
producing a sound speed map based on the obtained reception data for a sound speed map.
